(19) United States
(12) Patent Application Publication
(10) Pub. No.: US 2025/0017823 A1
(43) Pub. Date: Jan. 16, 2025

(54) HARD SHELL CAPSULES HAVING IMPROVED COLON RELEASE

(71) Applicant: Evonik Operations GmbH, Essen (DE)

(72) Inventors: Manfred Aßmus, Bickenbach (DE); Erna ROTH, Darmatadt (DE); Jessica BRUNS, Roßdorf (DE)

(73) Assignee: Evonik Operations Gmb, Essen (DE)

(21) Appl. No.: 18/577,125

(22) PCT Filed: Jun. 29, 2022
(86) PCT No.: PCT/EP2022/067846

§ 371 Date: Jan. 5, 2024

(30) Foreign Application Priority Data
Jul. 9, 2021 (EP) 21184696.9

(51) Int. Cl. A61J 3/07; A61K 47/32; A61K 47/36

(52) U.S. Cl. CPC A61J 3/077; A61K 47/32; A61K 47/36

(57) ABSTRACT

The invention refers to a process for preparing a polymer-coated hard shell capsule, wherein the hard shell capsule comprises a body and a cap, wherein in the closed state the cap overlaps the body either in a pre-locked state or in a final-locked state, wherein the hard shell capsule is provided in the pre-locked state and coated with a coating solution, suspension or dispersion comprising or consisting of
a) at least one (meth) acrylate copolymer a), comprising polymerized units of 5 to 25% by weight of methacrylic acid and 75 to 95% by weight of C1-to C4-alkylesters of methacrylic acid and/or C1-to C4-alkylesters of acrylic acid;
b) 1 to 25, preferably 5 to 18, % by weight, based on the total weight of the at least one (meth) acrylate copolymer a), of at least one alkali or ammonium salt of a saturated aliphatic monocarboxylic acid having 10 to 30 carbon atoms;
c) glycerol monostearate and/or glycerol distearate;
d) at least one plasticizer; and
e) optionally at least one additive;
   to obtain a coated hard shell capsule in the pre-locked state. Furthermore, the invention refers to a polymer-coated hard shell capsule obtained from the process according to the invention and the use of the polymer-coated hard shell capsule for delayed or sustained release.

### FIELD OF THE INVENTION

The invention refers to a process for preparing a polymer-coated hard shell capsule, wherein the hard shell capsule comprises a body and a cap, wherein in the closed state the cap overlaps the body either in a pre-locked state or in a final-locked state, wherein the hard shell capsule is provided in the pre-locked state and coated with a coating solution, suspension or dispersion comprising or consisting of
a) at least one (meth) acrylate copolymer a), comprising polymerized units of 5 to 25% by weight of methacrylic acid and 75 to 95% by weight of C1-to C4-alkylesters of methacrylic acid and/or C1-to C4-alkylesters of acrylic acid;
b) 1 to 25, preferably 5 to 18, % by weight, based on the total weight of the at least one (meth) acrylate copolymer a), of at least one alkali or ammonium salt of a saturated aliphatic monocarboxylic acid having 10 to 30 carbon atoms;
c) 2.5 to 7.5% by weight, based on the total weight of the at least one (meth) acrylate copolymer a) glycerol monostearate and/or glycerol distearate;
d) at least one plasticizer selected from the group consisting of alkyl citrates, alkyl phthalates, alkyl sebacates, diethyl sebacate, dibutyl sebacate, glycerol, polyethylene glycols, and polypropylene glycols; and
e) optionally at least one additive;
   to obtain a coated hard shell capsule in the pre-locked state. Furthermore, the invention refers to a polymer-coated hard shell capsule obtained from the process according to the invention and the use of the polymer-coated hard shell capsule for delayed or sustained release.

### BACKGROUND

The base material for hard shell capsules for drug delivery is commonly gelatin or hydroxypropyl methyl cellulose. When these capsules are not coated, they easily dissolve, independently of the pH value of the medium, in the stomach or intestine. Therefore, if a pH value dependent targeted drug release is desired, the hard shell capsules need a coating layer, whereby often (meth) acrylic polymers are used.

The object of the present invention was the provision of a very specific targeted drug release for hard shell capsules, namely a fast release in the colon. Thus, releasing the main portion of the drug shall only take place, if the colon is reached, i.e. at a pH value of 7.2 and in addition within about 1 h after the pH value of 7.2 has been reached. Accordingly, the object of the presently claimed invention is to provide a coating composition, which can accelerate the release of active ingredient at a pH of 7.2.

Suitable polymers and coatings for release after the stomach at pH value >6 have been disclosed for example under the tradename EUDRAGUARD® biotic and in WO 2011012163 A1. However, in this reference, even though capsules are mentioned in general, no example was provided for capsules and the release properties were only disclosed in view of tablets or pellets (e.g. page 26, second paragraph). Additionally, the coatings in this reference are considered to be suitable, if they start releasing at pH values of 6.0 or higher. No explicit coating for pH value 7.2 has been disclosed, all the examples, which were shown for pellets only, started already at pH value 6.0 and thus would be unsuitable to solve the present object.

Moreover, the inventors of the present invention surprisingly found that a release at pH value 7.2 for hard shell capsules can only be obtained by the specific coating composition in combination with the hard shell capsules according to the present invention. In this regard, it was particularly surprising that the same coating composition on pellets and capsules lead to different release characteristics. Therefore, a skilled person starting from the general teaching of WO 2011012163 A1, disclosing coatings for pellets, would not have come to the present invention.

### SUMMARY OF THE INVENTION

In a first aspect the invention refers to a process for preparing a polymer-coated hard shell capsule, wherein the hard shell capsule comprises a body and a cap, wherein in the closed state the cap overlaps the body either in a pre-locked state or in a final-locked state, wherein the hard shell capsule is provided in the pre-locked state and coated with a coating solution, suspension or dispersion comprising or consisting of
a) at least one (meth) acrylate copolymer a), comprising polymerized units of 5 to 25% by weight of methacrylic acid and 75 to 95% by weight of C1-to C4-alkylesters of methacrylic acid and/or C1-to C4-alkylesters of acrylic acid;
b) 1 to 25, preferably 5 to 18, % by weight, based on the total weight of the at least one (meth) acrylate copolymer a), of at least one alkali or ammonium salt of a saturated aliphatic monocarboxylic acid having 10 to 30 carbon atoms;
c) glycerol monostearate and/or glycerol distearate;
d) at least one plasticizer selected from the group consisting of alkyl citrates, alkyl phthalates, alkyl sebacates, diethyl sebacate, dibutyl sebacate, glycerol, polyethylene glycols, and polypropylene glycols; and
e) optionally at least one additive;
   to obtain a coated hard shell capsule in the pre-locked state.

In a second aspect the invention refers to a polymer-coated hard shell capsule obtained from the process according to the present invention.

In a third aspect the invention refers to the use of the polymer-coated hard shell capsule according to the present invention for delayed or sustained release.

### DETAILED DESCRIPTION OF THE INVENTION

### Hard Shell Capsules

Hard shell capsules for pharmaceutical or nutraceutical purposes are well known to a skilled person. A hard shell capsule is a two-piece encapsulation capsule comprising of the two capsule halves, called the body and the cap. The capsule body and cap material is usually made from a hard and sometimes brittle material. The hard shell capsule comprises a body and a cap. Body and cap are usually of a one end open cylindrical form with closed rounded hemispherical ends on the opposite end. The shape and size of the cap and body are such that the body can be pushed telescopically with its open end into the open end of the cap.

The body and the cap comprise a potential overlapping, matching area (overlap area) outside the body and inside the cap which partially overlap when the capsule is closed in the pre-locked state and totally overlap in the final-locked state. When the cap is partially slid over the overlapping matching area of the body the capsule is in the pre-locked state. When the cap is totally slid over the overlapping matching area of the body the capsule is in the final-locked state. The maintenance of the pre-locked state or of the final-locked state is usually supported by snap-in locking mechanisms of the body and the cap such as matching encircling notches or dimples, preferably elongated dimples.

Usually the body is longer than the cap. The outside overlapping area of the body can be covered by the cap in order to close or to lock the capsule. In the closed state the cap covers the outside overlap area of the body either in a pre-locked state or in a final-locked state. In the final-locked state the cap covers the outside overlap area of the body in total, in the pre-locked state the cap overlaps the outside overlapping area of the body only partially. The cap can be slid over the body to be fixed in usually one of two different positions in which the capsule is closed either in a pre-locked state or in a final-locked state.

Hard shell capsules are commercially available in different sizes. Hard shell capsules are usually delivered as empty containers with the body and cap already positioned in the pre-locked state and on demand as separate capsules halves, bodies and caps. The pre-locked hard shell capsules can be provided to a capsule-filling machine, which performs the opening, filling and closing of the capsule into the final-locked state. Usually hard shell capsules are filled with dry materials, for instance with powders or granules, or viscous liquids comprising a biologically active ingredient.

The cap and body are provided with closure means that are advantageous for the pre-locking (temporary) and/or final locking of the capsule. Therefore, elevated points can be provided on the inner wall of the cap and somewhat larger indented points are provided on the outer wall of the body, which are arranged so that when the capsule is closed the elevations fit into the indentations. Alternatively, the elevations can be formed on the outer wall of the body and the indentations on the inner wall of the cap. Arrangements in which the elevations or indentations arranged in a ring or spiral around the wall. Instead of the point-like configuration of the elevations and indentations, these may encircle the wall of the cap or body in an annular configuration, although advantageously recesses and openings are provided which enable an exchange of gases into and out of the capsule interior. One or more elevations can be provided in an annular arrangement around the inner wall of the cap and the outer wall of the body such that, in the final-locked position of the capsule, an elevation on the cap is located adjacent to an elevation on the body. Sometimes elevations are formed on the outside of the body close to the open end and indentations are formed in the cap close to the open end such that the elevations on the body latch into the indentations in the cap in the final-locked position of the capsule. The elevations can be such that the cap can be opened in the pre-locked state at any time without damage to the capsule or, alternatively, so that once it has been closed the capsule cannot be opened again without destroying it. Capsules with one or more such latching mechanisms (latches) (for example two encircling grooves) are preferred. More preferred are capsules with at least two such latching means which secure the two capsule parts to different degrees. In a part of this kind, a first latching (dimples or encircling notches) means can be formed close to the openings in the capsule cap and the capsule body and a second latching (encircling notches) can be shifted somewhat further towards the closed end of the capsule parts. The first latching means secure the two capsule parts less strongly than the second does. This variant has the advantage that after the production of the empty capsules the capsule cap and capsule body can initially be pre-locked joined together using the first latching mechanism. In order to fill the capsule, the two capsule parts are then separated again. After filling, the two capsule parts are pushed together until the second set of latches firmly secures the capsule parts in a final-locked state.

Preferably, the body and the cap of the hard shell capsule are comprising each encircling notches and/or dimples in the area, where the cap can be slid over the body. Encircling notches of the body and dimples of the cap match to each other to provide a snap-in or snap into-place mechanism. The dimples can be circular or elongated (oval) in the longitudinal direction. Encircling notches of the body and encircling notches of the cap (closely matched rings) also match to each other to provide a snap-in or snap into-place mechanism. This allows the capsule to be closed by a snap-into-place mechanism either in a pre-locked state or in a final-locked state.

Preferably, matching encircling notches of the body and elongated dimples of the cap are used to fix the body and the cap to each other in the pre-locked state. Matching encircling notches of the body and the cap are preferably used to fix or lock the body and the cap to each other in the final-locked state.

The area, where the cap can be slid over the body can be called the overlapping area of the body and the cap or briefly the overlap area. If the cap overlaps the body only partially, maybe to 20 to 90 or 60 to 85% of the overlap area, the hard shell capsule is only partially closed (pre-locked). Preferably, in the presence of a locking mechanism, like matching encircling notches and/or dimples in body and cap, the partially closed capsule can be called pre-locked. When the capsule is polymer-coated in the pre-locked state the coating will cover the completely outer surface including that part of the overlap area of the body and cap that is not overlapped by the cap in this pre-locked state. When the capsule is polymer-coated in the pre-locked state and then closed to the final-locked state the coating of that part of the overlap area of the body and cap that was not overlapped by the cap in the pre-locked state will then become covered by the cap. The presence of that part of the coating which is then enclosed in the final-locked state between the body and the cap is sufficient for the hard shell capsule to be tightly sealed.

If the cap overlaps the body the total overlapping area of the body, the hard shell capsule is finally closed or in the final-locked state. Preferably, in the presence of a locking mechanism, like matching encircling notches and/or dimples in body and cap, the finally closed capsule can be called final-locked.

Usually dimples are preferred for the fixing the body and the cap in the pre-locked state. As a non-binding rule the matching area of dimples is smaller than the matching area of encircling notches. Thus snapped-in dimples can be snapped-out again by applying less forces than those that would be necessary to snap-out a snapped-in fixation by matching encircling notches.

The dimples of the body and cap are located in the area, where the cap can be slid over the body match to each other in the pre-locked state by a snap in or snap into-place mechanism. There can be for example 2, 4, or preferably 6 notches or dimples located distributed circular around the cap.

Usually the dimples of the cap are and the encircling notches of the body in the area, where the cap can be slid over the body match to each other so that they that allow the capsule to be closed by a snap-into-place mechanism in the pre-locked state. In the pre-locked state, the hard shell capsule can be re-opened manually or by a machine without damaging, because the forces needed to open are comparatively low. Thus, the “pre-locked state” is sometimes designated also as “loosely capped”.

Usually the encircling notches or matching locking rings of the body and the cap in the area, where the cap can be slid over the body match to each other so that they that allow the capsule to be closed by a snap-into-place mechanism in the final-locked state. In the final-locked state, the hard shell capsule cannot or can only hardly be re-opened manually or by a machine without damaging, because the forces needed to open are comparatively high.

Usually dimples and the encircling notches are formed in the capsule body or capsule cap. When the capsule parts provided with these elevations and indentations are fitted into one another, ideally defined uniform gaps of from 10 microns to 150 microns, more particularly 20 microns to 100 microns, are formed along the contact surface between the capsule body and the capsule cap placed thereon.

Preferably, the body of the hard shell capsule comprises a tapered rim. The tapered rim prevent the rims of the body and the cap to collide and becoming damaged when the capsule is closed manually or by a machine.

In contrast to a hard shell capsule, a soft shell capsule is a welded one piece encapsulation capsule. A soft gel capsule is often made from blow molded soft gelling substances and is usually filled with liquids comprising a biologically active ingredient by injection. The invention is not concerned with welded soft shell one piece encapsulation capsules.

### Sizes of Hard Shell Capsules

A closed, final-locked hard shell capsule can have a total length in the range from about 5 to 40 mm. The diameter of the cap can be in the range from about 1.3 to 12 mm. The diameter of the body can be in the range from about 1.2 to 11 mm. The length of the cap can be in the range from about 4 to 20 mm and that of the body in the range from 8 to 30 mm. The fill volume can be between about from 0.004 to 2 ml. The difference between the pre-locked length and the final-locked length can be about 1 to 5 mm.

Capsules can be divided into standardized sizes for example from sizes 000 to 5. A closed capsule of size 000 has, for example, a total length of about 28 mm with an outer diameter of the cap of about 9.9 mm and an outer diameter of the body of about 9.5 mm. The length of the cap is about 14 mm, that of the body about 22 mm. The fill volume is about 1.4 ml.

A closed capsule of size 5 has, for example, a total length of about 10 mm and an outer diameter of the cap of about 4.8 mm and an outer diameter of the body of about 4.6 mm. The length of the cap is about 5.6 mm, that of the body about 9.4 mm. The fill volume is about 0.13 ml.

A size 0 capsule may show a length of about 23 to 24 mm in the pre-locked state and of about 20.5 to 21.5 mm in the final-locked state. Thus, the difference between the pre-locked length and the final-locked length can be about 2 to 3 mm.

### Coated Hard Shell Capsule

The invention is concerned with a polymer-coated hard shell capsule, obtained by the process as described herein.

### Material of the Body and the Cap

The base material of the body and the cap can be selected from hydroxypropyl methyl cellulose, starch, gelatin, pullulan and a copolymer of C1-to C4-alkylester of (meth)acrylic acid and (meth)acrylic acid. Preferred are hard shell capsules where body and cap are comprising or consisting of HPMC or gelatin, most preferred is HPMC because of its good adhesion properties for the polymer coating.

### Component a): The (Meth)Acrylate Copolymer

The solution, suspension or dispersion comprises at least one (meth)acrylate copolymer a).

The (meth)acrylate copolymer a) comprises polymerized units of 5 to 25% by weight of methacrylic acid and 75 to 95% by weight of C1-to C4-alkylesters of methacrylic acid and/or C1- to C4-alkylesters of acrylic acid. The (meth)acrylate copolymer a) can be present in the form of polymeric particles. Preferably the monomers can add up to 100%.

C1-to C4-alkyl esters of acrylic or methacrylic acid are in particular methyl methacrylate, ethyl methacrylate, butyl methacrylate, methyl acrylate, ethyl acrylate and butyl acrylate.

The (meth)acrylate copolymer a) can be obtained from an emulsion polymerization process, wherein the whole amount of all of the monomers are charged and are polymerized simultaneously in one step to polymeric particles. As a result of the process, the polymeric particles show a unique distribution of the monomers, especially the polymerized units of methacrylic acid, can be deemed constant, from the center (inside) and surface (outside) of the particles.

The polymeric particles can be present in aqueous dispersed form or in the form of a re-dispersible powder gained by drying from an aqueous dispersion comprising the polymeric particles.

Preferably the (meth)acrylate copolymer a) comprises a monomer composition comprising polymerized units of 10 to 30% by weight of methyl methacrylate, 50 to 70% by weight of methyl acrylate and 5 to 15% by weight of methacrylic acid. Preferably the monomers may add up to 100%.

A typical (meth)acrylate copolymer a) may be EUDRAGIT® FS 30 D, a well-known commercially available (meth)acrylate copolymer product for pharmaceutical applications in the form of a 30% by weight aqueous dispersion. The copolymer is polymerized from 10% by weight of methacrylic acid, 65% by weight of methyl acrylate, and 25% by weight of methyl methacrylate. The EUDRAGUARD® biotic has same chemical composition as EUDRAGIT® FS 30 D.

The specific dissolution pH-value of the (meth)acrylate copolymer a), especially of the EUDRAGIT® FS 30 D polymer, is from about pH 7.0 to pH 7.2. There is no considerable dissolution below pH 7.0, for instance at pH 6.8.

### Process for Preparing a (meth)acrylate Copolymer a)

The (meth)acrylate copolymer a) may be prepared in a manner known in the art by free-radical polymerization of the monomers as described, for example, in EP 0 704 207 A2 and EP 0 704 208 A2. The (meth)acrylate copolymer a) can be prepared by conventional processes of free-radical polymerization continuously by batch processes, for example by emulsion polymerization in the presence of free-radical forming initiators and, where appropriate, regulators to adjust the molecular weight undiluted, in solution, by bead polymerization or in emulsion. The average molecular weight Mw (weight average, determined for example by measuring the solution viscosity) can be for example in the range from 80 000 g/mol to 1 000 000 g/mol.

Emulsion polymerization in aqueous phase in the presence of water-soluble initiators and, preferably anionic, emulsifiers is preferred. The weight-average size (radius) of the resulting polymeric particles is usually in the range from 50 to 500, preferably 80 to 300 nm, thus ensuring a viscosity below 1000 mPa·s, which is favourable for processing techniques. The particle size can be determined by laser diffraction, e.g. using the Mastersizer 2000 (from Malvern Inc.). In the case of bulk polymerization, the copolymer can be obtained in solid form by crushing, extrusion, granulation or hot cut.

The (meth)acrylate copolymer a) can be obtained in a manner known in the art by free-radical bulk, solution, bead or emulsion polymerization. It can be brought before processing to the appropriate particle size range by suitable grinding, drying or spraying processes. This can be achieved by simple crushing of extruded and cooled pellets or hot cut. The use of polymer powder may be advantageous, especially for mixing with other powders or liquids. Typical equipment suitable for producing of powders is well known to those skilled in the art, e.g. air jet mill, pinned disc mill, compartment mill. It is possible, where appropriate to include appropriate sieving steps. A suitable mill for industrial large quantities is, for example, an opposed jet mill (Multi No. 4200) operated with a gauge pressure of about 6 bar.

An emulsion polymerization process can advantageously be carried out by the monomer emulsion feed process or the monomer feed process, respectively, in a polymerization reactor. For this, water is heated to the reaction temperature in a polymerization reactor. Surfactants and/or initiators may be added at this stage. The whole amounts of all monomers may be charged into the reactor before adding the initiator. This method is often referred to as “batch emulsion process”.

Emulsifiers which may be used are especially anionic and non-ionic surfactants. The amount of emulsifier used is generally not more than 5% by weight, preferably 0.1 to 3% by weight, based on the weight of the monomers. Typical emulsifiers are for example alkyl sulfates (e.g. sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and others.

Beside those polymerization initiators conventionally used in emulsion polymerization (e.g. per-compounds, such as ammonium peroxodisulfate (APS)) redox systems, such as sodium disulphite-APS-iron, can be applied. Also, water-soluble azo initiators may be applied and/or a mixture of initiators can be used. The amount of initiator is usually between 0.005 to 0.5, preferably 0.01 to 0.3% by weight, based on the weight of the monomers.

A chain transfer agent may be added to improve the process stability and the reproducibility of the molecular weight (Mw). A typical amount of chain transfer agent may be 0.05 to 1% by weight based on monomer weight. A typical chain transfer agent may be, for example, thioglycolic acid 2-ethyl hexyl ester (TGEH) or n-dodecyl mercaptan (nDDM). However, the chain transfer agent may be omitted in some cases, without affecting the properties according to the invention.

A typical emulsion polymerization broth may comprise the monomers and water at a typical ratio by weight of about 3 to 7 as main components and 0.005 to 0.5% by weight of one more polymerization initiator(s), 0.05 to 1% by weight of a chain transfer agent(s), less than 5% by weight or 0.1 to 3.0% by weight of an emulsifier and 0 to 0.5% by weight of an antifoam agent, wherein all components may add up to 100%.

The polymerization temperature depends on the initiators within certain limits. For example, if APS is used, it is advantageous to operate in the range from 60 to 90° C.; if redox systems are used it is also possible to polymerize at lower temperatures, for example at 30° C.

At the end of the process the reactor content is usually allowed to cool down, for instance to 20 to 25° C. and the resulting dispersion may be filtered, for instance through a 250 μm gaze.

The average particle size (D50) of the polymeric particles produced in the emulsion polymerization can range from 50 to 500, preferably 80 to 300 nm. The average particle size of the polymer particles can be determined by methods well known to a skilled person, for instance by the method of laser diffraction. The particle size can be determined by laser diffraction, using a Mastersizer 2000 (Malvern). The values can be indicated as particle radius rMS [nm], which is half of the median of the volume-based particle size distribution d(v,50).

The dispersion can also be dried to a powder or granulate, preferably by spray drying, spray granulation, freeze drying, coagulation or extrusion. Thus, a solid powder or granulate can be obtained, which offers certain advantages with regard to handling and logistics. The dry powder or granulate may be used as polymeric binder for matrix dosage forms.

The dried polymerizate may then be transferred into a coating suspension by re-dispersing the solid in water, e.g. (where required) by the use of a high shear mixer.

### Aqueous Dispersion of the (meth)acrylate Copolymer a)

The (meth)acrylate copolymer a) is usually gained from an emulsion polymerization process in the form of an aqueous dispersion or commercially available as such a dispersion (EUDRAGIT® FS 30 D), for instance at a polymer concentration of about 30% by weight. The components b) and (c) and/or d)) and optionally at least one additive may then be added to the aqueous dispersion for further processing in applications as coating or binding agent.

### Powder or Granulate

The (meth)acrylate copolymer a) may be converted from an aqueous dispersion to a dry form, preferably to a powder or a granulate, by spray drying, spray granulation, spray agglomeration, freeze drying, coagulation or extrusion of the aqueous dispersion. The resulting granulate or powder may have a particle size D50 in the range from about 0.01 to 5 mm. Powder can have a particle size D50 in the range from about 0.01 up to less than 0.5 mm. Granulates can have a particle size D50 in the range from about 0.5 mm up to 5 mm. The average particle size of granulates is preferably determined by well-known sieving methods. The particle size D50 of powder is preferably determined by laser diffraction. The dry form of (meth)acrylate copolymer a) can be used for re-dispersion to an aqueous dispersion or alternatively for dry mixing with the components b) and (c) and/or d)) to gain a (ready to use) composition in dry form as disclosed. The dry form can be converted again to an aqueous dispersion, optionally the at least one additive can then be added for further processing in applications as coating or binding agent.

### Component b): Monocarboxylic Acid

The composition comprises 1 to 25, preferably 5 to 15% by weight, based on the total weight of the at least one (meth)acrylate copolymer a), of an alkali or ammonium salt of a saturated aliphatic monocarboxylic acid having 10 to 30 carbon atoms.

The alkali or ammonium salt of the saturated aliphatic monocarboxylic acid having 10 to 30 carbon atoms may be selected from alkali or ammonium salts of the following monocarboxylic acids: decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid, and triacontanoic acid (melissic acid, C30).

Preferably, the alkali salt of a saturated aliphatic monocarboxylic acid having 10 to 30 carbon atoms is sodium stearate.

### Glidants

Glidants usually have lipophilic properties. They prevent agglomeration of cores during film formation of the film forming polymers.

The coating solution, suspension or dispersion comprises glycerol monostearate and/or glycerol distearate.

The coating solution, suspension or dispersion can comprise at least one further glidant different from glycerol monostearate and glycerol distearate, which is preferably selected from silica, for example commercially available under the tradenames RXCIPIENTS® GL100 or RXCIPIENTS® GL200, ground silica, fumed silica, kaolin calcium silicate, magnesium silicate, colloidal silicone dioxide, talc, stearate salts selected from the group consisting of calcium stearate, magnesium stearate, and zinc stearate, sodium stearyl fumarate, starch, stearic acid, preferably talc, magnesium stearate, and colloidal silicon dioxide or mixtures thereof.

Standard proportions for use of total weight of glidants in the inventive coating range between 0.5 and 100% by weight, preferably 3 to 75% by weight, more preferably 5 to 50% by weight, most preferably 5 to 30% by weight, based on the total weight of the at least one (meth)acrylate copolymer.

### Plasticizers

The polymer coating of the hard shell capsule comprises at least one plasticizer. Plasticizers achieve through physical interaction with a polymer a reduction in the glass transition temperature and promote film formation, depending on the added amount. Suitable substances usually have a molecular weight of between 100 and 20,000 g/mol and comprise one or more hydrophilic groups in the molecule, e.g. hydroxyl, ester or amino groups.

Examples of suitable plasticizers selected from the group consisting of alkyl citrates, alkyl phthalates, alkyl sebacates, diethyl sebacate, dibutyl sebacate, glycerol, polyethylene glycols, and polypropylene glycols. Preferred plasticizers are triethyl citrate (TEC), glycerol, acetyl triethyl citrate (ATEC), diethyl sebacate, dibutyl sebacate (DBS), polyethylene glycols, and polypropylene glycols or mixtures thereof.

In another preferred embodiment, the alkyl citrate according to any one of the preceding embodiment is triethyl citrate.

Addition of the plasticizers to the formulation can be carried out in a known manner, directly, in aqueous solution or after thermal pre-treatment of the mixture. It is also possible to employ mixtures of plasticizers. The polymer coating of the hard shell capsule may comprise one or more plasticizers, preferably up to 60, up to 30, up to 25, up to 20, up to 15, up to 10, up to 5, or up to 2 wt.-%, based on the total weight of the at least one (meth)acrylate copolymer.

### Coating Layer

The coating layer obtained by coating the hard shell capsule with a method according to the present invention can comprise 10% or more, 20% or more, 30% or more, 40% or more, 50% or more, 60% or more, 70% or more, 80% or more, 90% or more by weight or 95% or more by weight of the at least one (meth)acrylate copolymer. The coating layer can comprise 10-100, 10-90, 12-80, 15-80, 18-80, 20-80 or 40 to 80% by weight of the at least one (meth)acrylate copolymer.

### Amount and Thickness of the Coating Layer

For hard shell capsules, the amount of the coating layer should not be too high. If the amount of coating layer applied is too high this may result in difficulties to process the polymer-coated pre-locked hard shell capsules subsequently in a capsule-filling machine. If the amount of coating layer is less than 8 mg/cm², for instance 1 to 8 mg/cm² or 1 to 7 mg/cm² or 1 to 7 mg/cm² or 1 to 6 mg/cm² or 1 to 5 mg/cm² or 1 to 4 mg/cm² usually no problem with standard capsule-filling machines without modification will occur. In the range from 4 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 8 mg/cm².

For a hard shell capsule of size #0, the amount of the coating layer should not be too high. If the amount of coating layer applied is too high this may result in difficulties to process the polymer-coated pre-locked hard shell capsules subsequently in a capsule-filling machine. If the amount of coating layer is less than 5 mg/cm², for instance 1 to 4 mg/cm² usually no problem with standard capsule-filling machines without modification will occur. In the range from 4 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 8 mg/cm².

For a hard shell capsule of size #1, the amount of the coating layer should not be too high. If the amount of coating layer applied is too high this may result in difficulties to process the polymer-coated pre-locked hard shell capsules subsequently in a capsule-filling machine. If the amount of coating layer is less than 4 mg/cm², for instance 1 to 3.5 mg/cm² usually no problem with standard capsule-filling machines without modification will occur. In the range from 3.5 and up to about 8 mg/cm² capsule-filling machines can still be used, however the forms for the bodies and the caps should be adjusted to be somewhat wider. Such an adjustment can be easily performed by a mechanical engineer. Thus capsule-filling machines can be advantageously used within a range of an amount of coating layer from about 1 to about 8 mg/cm².

For a hard shell capsule of size #3, the amount of the coating layer should not be too high. If the amount of coating layer applied is too high this may result in difficulties to process the polymer-coated pre-locked hard shell capsules subsequently in a capsule-filling machine. If the amount of coating layer is less than 3 mg/cm², for instance 1 to 2.5 mg/cm² usually no problem with standard capsule-filling machines without modification will occur. In the range from 2.5 and up to about 6 mg/cm² capsule-filling machines can still be used, however the forms for the bodies and the caps should be adjusted to be somewhat wider. Such an adjustment can be easily performed by a mechanical engineer. Thus, capsule-filling machines can be advantageously used within a range of an amount of coating layer from about 1 to about 6 mg/cm².

Above 8 mg/cm² and up to about 20 mg/cm² careful manual opening of the polymer-coated hard shell capsule, filling and closing to the pre-locked state may still be possible without causing damage to the polymer coating. If the coating layer is thicker than the gap between the uncoated body and the cap, the coated pre-locked capsules can result in difficulties to be closed without damaging the applied coating as the cap can hardly slide over the body to the final-locked state anymore.

If the amount of coating layer applied is too high there will be also an assembly of too much coating layer at the rim of the cap where the gap between body and cap is in the pre-locked state. This may result after drying in fissures of the coating layer when the coated pre-locked hard shell capsule is opened manually or in a machine. The fissures may result in a later leakage of the capsule. Finally, a too thick coating may result in difficulties or make it impossible to close the opened coated hard shell capsule to the final-locked state since the coating layer is thicker than the gap in the overlapping area between the body and the cap.

As a general rule the coating layer on the hard shell capsule can preferably be applied in an amount (=a total weight gain) of 0.7 to 20, 1.0-18, 2 to 10, 4 to 8, 1.0 to 8, 1.5 to 5.5, 1.5 to 4 mg/cm².

As a general rule the coating layer on the hard shell capsule can have an average thickness of about 5 to 100, 10 to 50, 15 to 75 μm.

As a general rule the coating layer on the hard shell capsule can be applied in an amount of 5 to 50, preferably 8-40% dry weight in relation to the weight of the pre-locked capsule.

With this guidance a skilled person will be able to adjust the amounts of the coating layer in a range between too low and too high.

### Additives

Additives according to the present invention are preferably biologically active ingredients and excipients, which are well known to a skilled person and often formulated along with the biologically active ingredient contained in the coated hard shell capsule and/or with the polymer coating of the hard shell capsule as disclosed and claimed herein. All excipients used must be toxicologically safe and be used in pharmaceuticals or nutraceuticals without risk for patients or consumers.

The dosage form may comprise excipients, preferably pharmaceutically or nutraceutically acceptable excipients, selected from the group of antioxidants, brighteners, binding agents, emulsifier, flavouring agents, flow aids, fragrances, penetration-promoting agents, pigments, pore-forming agents or stabilizers or combinations thereof. The pharmaceutically or nutraceutically acceptable excipients can be comprised in the core and/or in the coating layer comprising the polymer as disclosed. A pharmaceutically or nutraceutically acceptable excipient is an excipient, which is allowed to be used for the application in the pharmaceutical or nutraceutical field.

The coating layer may comprise up to 90, up to 80, up to 70, up to 50, up to 60, up to 50, up to 40, up to 30, up to 20, up to 10, up to 5% up to 3%, up to 1% by weight or not any (0%) additives at all, respectively pharmaceutically or nutraceutically acceptable excipients.

### Emulsifiers

At least one emulsifier can be additionally present. In general, all known emulsifiers are suitable. Preferred are non-ionic emulsifiers, in particular emulsifier having an HLB>10. The HBL Value can be determined according to Griffin, William C. (1954), “Calculation of HLB Values of Non-Ionic Surfactants” (PDF), Journal of the Society of Cosmetic Chemists, 5 (4): 249-56.

The at least one emulsifier is preferably selected from polyglycosides, alcohols, sugar and sugar derivatives, polyethers, amines, polyethylene derivatives, alkyl sulfates (e.g. sodium dodecyl sulfate), alkyl ether sulfates, dioctyl sodium sulfosuccinate, polysorbates (e.g. polyoxyethylene (20) sorbitan monooleate), nonylphenol ethoxylates (nonoxynol-9) and mixtures thereof.

The at least one emulsifier is preferably selected from alkyl polyglycosides, decyl glucoside, decyl polyglucose, lauryl glucoside, octyl glucoside, N-octyl beta-D-thioglucopyranoside, cetostearyl alcohol, cetyl alcohol, stearyl alcohol, polyoxyethylene cetostearyl alcohol, cetylstearyl alcohol, oleyl alcohol, polyglyceryl-6-dioleate, glyceryl stearate citrate, polyglyceryl-3 caprate, polyglyceryl-3 diisostearate, glyceryl isostearate, polyglyceryl-4-isostearate, glyceryl monolinoleate, dicaprylyl carbonate, alcohol polyglycol ether, polyethylenglycolether of cetearylalcohols (n=20), polyethylene glycol-6 stearate, glycol stearate, polyethylene glycol-32 stearate, polyethylene glycol-20 stearate, fatty alcohol polyglycol ether, polyethylene glycol-4 laurate, polyethylene glycol isocetyl ether (n=20), polyethyleneglycol-32 (Mw 1500 g/mol) mono-and diesters of lauric acid (C12), nonaethylene glycol, polyethylene glycol nonylphenyl ether, octaethylene glycol monododecyl ether, pentaethylene glycol monododecyl ether, polyethylene glycol macrocetyl ether, polyethylene glycol esters of palmitic (C16) or stearic (C18) or caprylic acids, polyoxyethylene fatty ether derived from stearyl alcohols like BRIJ S2, polyoxyethylene oxypropylene stearate, macrogol stearyl ether (20), diethylaminoethyl stearate, polyethylene glycol stearate, sucrose distearate, sucrose tristearate, sorbitan monostearate, sorbitan tristearate, mannide monooleate, octaglycerol monooleate, sorbitan dioleate, polyricinoleate, polysorbate like polysorbate 20 and Polyoxyethylene (20) sorbitan monooleate (polysorbate 80), sorbitan, sorbitan monolaurate, sucrose cocoate, glycereth-2 cocoate, ethylhexyl cocoate, polypropylene glycol-3 benzyl ether myristate, sodium myristate, gold sodium thiomalate, polyethylene glycol 8 laurate, polyethylene-4 dilaurate, from α-Hexadecyl-ω-hydroxypoly (oxyethylene), cocamide diethanolamine, N-(2-hydroxyethyl)dodecanamide, octylphenoxypolyethoxyethanol, maltoside, 2,3-Dihydroxypropyl dodecanoate, 3-[(3R,6R,9R,12R, 15S,22S,25S,30aS)-6,9,15,22-Tetrakis (2-amino-2-oxoethyl)-3-(4-hydroxybenzyl)-12-(hydroxymethyl)-18-(11-methyltridecyl)-1,4,7, 10, 13, 16,20,23,26-nonaoxotriacontahydropyrrolo[1,2-g][1,4,7,10,13,16,19,22,25]nonaazacyclooctacosin-25-yl]propenamide, 2-{2-[2-(2-{2-[2-(2-{2-[2-(4-nonylphenoxy)ethoxy]ethoxy}ethoxy) ethoxy]ethoxy}ethoxy) ethoxy]ethoxy}ethanol, oxypolyethoxydodecane, poloxamers like poloxamer 188 (Pluronic F-68) and poloxamer 407, propylene glycol monocaprylate, type I (Capryol PGMC), polyethoxylated tallow amine, polyglycerol, polyoxyl 40 hydrogenated castor oil, surfactin, 2-[4-(2,4,4-trimethylpentan-2-yl)phenoxy]ethanol, carbomer, sodium carbomer, carboxymethylcellulose calcium, carrageenan, cholesterol, deoxycholic acid, phospholipids like egg phospholipids, gellan gum, lanolin, capric acid, waxes like Polawax NF, Polawax A31 or Ceral PW, ester gum, dea-cetyl phosphate, soya lecithin, sphingomyelins, sodium phosphate, sodium lauroyl lactylate, lanolin, Oxirane methyl-polymer with oxirane monobutyl ether, 1,2-dierucoylphosphatidylcholine, dimethicone end-blocked with an average of 14 moles of propylene oxide, laurylmethicone copolyol, lauroglycol 90, white mineral oil like Amphocerine KS, dispersion of acrylamide/sodium acryloyldimethyl taurate copolymer in isohexadecane, and sodium polyacrylate or mixtures thereof. Preferred are macrogol stearyl ether (20) and polysorbate 80.

### Fillers

Standard fillers can usually be added to the inventive formulation during processing to coating and binding agents. The quantities introduced and the use of standard fillers in pharmaceutical coatings or over layers is familiar to those skilled in the art. Examples of standard fillers are release agents, pigments, stabilizers, antioxidants, pore-forming agents, penetration-promoting agents, brighteners, fragrances or flavoring agents. They are used as processing adjuvants and are intended to ensure a reliable and reproducible preparation process as well as good long-term storage stability, or they achieve additional advantageous properties in the pharmaceutical form. They are added to the polymer formulations before processing and can influence the permeability of the coatings. This property can be used if necessary, as an additional control parameter.

### Pigments

Only rarely a pigment is added in soluble form. As a rule, pigments, such as aluminum oxide or iron oxide pigments are used in dispersed form. Titanium dioxide is used as a whitening pigment. Standard proportions for use of pigments are between 10-200, 20-200% by weight relative to the total weight of the at least one polymer in the coating layer. Proportions up to 200% by weight based on the total weight of the at least one polymer can be easily processed.

In a particularly advantageous embodiment, the pigment is used directly in concentrated form as an additional outer layer, a so called top coat. Application takes place in the form of powder or by spraying from aqueous suspension with 5 to 35% (w/w) solid content. The necessary concentration is lower than for incorporation into the polymer layer and amounts to 0.1 to 2% by weight relative to the weight of the pharmaceutical form.

### Biologically Active Ingredient

A biologically active ingredient is preferably a pharmaceutically active ingredient and/or a nutraceutically active ingredient and/or a cosmetically active ingredient. Even though it is possible that certain biologically active ingredients are contained in the coating layer, it is preferred that the biologically active ingredient is contained in the fill-in. In particular, biologically active ingredients like nucleic acids require delivery vehicles, for example a liposome, lipid nanoparticle or an appropriate polymer-based carrier, and in this case the biologically active ingredient is only contained in the fill-in.

### Pharmaceutically or Nutraceutically Active Ingredients

The invention is preferably useful for delayed release or sustained release formulated pharmaceutical or nutraceutical dosage forms with a fill-in of pharmaceutically or nutraceutically active ingredients.

Suitable therapeutic and chemical classes of pharmaceutically active ingredients which members can be used as fill-in for the described polymer-coated hard shell capsules are for instance: analgesics, antibiotics or anti-infectives, antibodies, antiepileptics, antigens from plants, antirheumatics, benzimidazole derivatives, beta-blocker, cardiovascular drugs, chemotherapeutics, CNS drugs, digitalis glycosides, gastrointestinal drugs, e.g. proton pump inhibitors, enzymes, hormones, liquid or solid natural extracts, nucleic acids, oligonucleotides, peptides, hormones, proteins and its corresponding (metal) salts, therapeutic bacteria, urology drugs or vaccines, the latter may use appropriate lipid or polymer based delivery vehicles, for example, lipid nanoparticles, liposomes or charge-altering releasable transporters (CARTs).

In an embodiment the pharmaceutically active ingredient is a nucleic acid, more preferably a nucleic acid agent can be DNA, RNA, or combinations thereof. In some embodiments, a nucleic acid agent can be an oligonucleotide and/or polynucleotide. In some embodiments, a nucleic acid agent may be an oligonucleotide and/or modified oligonucleotide (including, but not limited to, modifications through phosphorylation); an antisense oligonucleotide and/or modified antisense oligonucleotide (including, but not limited to, modifications through phosphorylation). In some embodiments, a nucleic acid agent can comprise cDNA and/or genomic DNA. In some embodiments, a nucleic acid agent can comprise non-human DNA and/or RNA (e.g., viral, bacterial, or fungal nucleic acid sequences). In some embodiments, a nucleic acid agent can be a plasmid, cosmid, gene fragment, artificial and/or natural chromosome (e.g., a yeast artificial chromosome), and/or a part thereof. In some embodiments, a nucleic acid agent can be a functional RNA (e.g., a mRNA, a tRNA, an rRNA and/or a ribozyme). In some embodiments, a nucleic acid agent can be an RNAi-inducing agent, small interfering RNA (siRNA), short hairpin RNA (shRNA), and/or microRNA (miRNA). In some embodiments, a nucleic acid agent can be a peptide nucleic acid (PNA). In some embodiments, a nucleic acid agent can be a polynucleotide comprising synthetic analogues of nucleic acids, which may be modified or unmodified. In some embodiments, a nucleic acid agent can comprise various structural forms of DNA including single-stranded DNA, double-stranded DNA, supercoiled DNA and/or triple-helical DNA; Z-DNA; and/or combinations thereof. Further suitable nucleic acids are for example disclosed in WO 2012103035 A1, which are incorporated by reference.

Further examples of drugs that can be used as fill-in for the described polymer-coated hard shell capsules are for instance acamprosat, aescin, amylase, acetylsalicylic acid, adrenalin, 5-amino salicylic acid, aureomycin, bacitracin, balsalazine, beta carotene, bicalutamid, bisacodyl, bromelain, bromelain, budesonide, calcitonin, carbamacipine, carboplatin, cephalosporins, cetrorelix, clarithromycin, chloromycetin, cimetidine, cisapride, cladribine, clorazepate, cromalyn, 1-deaminocysteine-8-D-arginine-vasopressin, deramciclane, detirelix, dexlansoprazole, diclofenac, didanosine, digitoxin and other digitalis glycosides, dihydrostreptomycin, dimethicone, divalproex, drospirenone, duloxetine, enzymes, erythromycin, esomeprazole, estrogens, etoposide, famotidine, fluorides, garlic oil, glucagon, granulocyte colony stimulating factor (G-CSF), heparin, hydrocortisone, human growth hormon (hGH), ibuprofen, ilaprazole, insulin, Interferon, Interleukin, Intron A, ketoprofen, lansoprazole, leuprolidacetat lipase, lipoic acid, lithium, kinin, memantine, mesalazine, methenamine, milameline, minerals, minoprazole, naproxen, natamycin, nitrofurantion, novobiocin, olsalazine, omeprazole, orothates, pancreatin, pantoprazole, parathyroidhormone, paroxetine, penicillin, perprazol, pindolol, polymyxin, potassium, pravastatin, prednisone, preglumetacin progabide, pro-somatostatin, protease, quinapril, rabeprazole, ranitidine, ranolazine, reboxetine, rutosid, somatostatin streptomycin, subtilin, sulfasalazine, sulphanilamide, tamsulosin, tenatoprazole, thrypsine, valproic acid, vasopressin, vitamins, zinc, including their salts, derivatives, polymorphs, isomorphs, or any kinds of mixtures or combinations thereof.

It is evident to a skilled person that there is a broad overlap between the terms pharmaceutically and nutraceutically active ingredients, excipients and compositions respectively a pharmaceutical or a nutraceutical dosage form. Many substances listed as nutraceuticals may also be used as pharmaceutically active ingredients. Depending on the specific application and local authority legislation and classification, the same substance can be listed as a pharmaceutically or a nutraceutically active ingredient respectively a pharmaceutical or a nutraceutical composition or even both.

Nutraceuticals are well known to the skilled person. Nutraceuticals are often defined as extracts of foods claimed to have medical effects on human health. Thus, nutraceutically active ingredients may display pharmaceutical activities as well: Examples for nutraceutically active ingredients can be resveratrol from grape products as an antioxidant, soluble dietary fiber products, such as psyllium seed husk for reducing hypercholesterolemia, broccoli (sulphane) as a cancer preservative, and soy or clover (isoflavonoids) to improve arterial health. Thus, it is clear that many substances listed as nutraceuticals may also be used as pharmaceutical active ingredients.

Typical nutraceuticals or nutraceutically active ingredients that can be used as fill-in for the described polymer-coated hard shell capsules may also include probiotics and prebiotics. Probiotics are living microorganisms believed to support human or animal health when consumed. Prebiotics are nutraceuticals or nutraceutically active ingredients that induce or promote the growth or activity of beneficial microorganisms in the human or animal intestine.

Examples for nutraceuticals are resveratrol from grape products, omega-3-fatty acids or (pro-) anthocyanidins, e.g. from blueberries, bilberries or black currants, as antioxidants, soluble dietary fiber products, such as psyllium seed husk for reducing hypercholesterolemia, broccoli (sulphane) as a cancer preservative, and soy or clover (isoflavonoids) to improve arterial health. Other nutraceuticals examples are flavonoids, alpha-linoleic acid from flax seed, beta-carotene from marigold petals or anthocyanins from fruits, e.g. from various berries, vegetables and grains. Sometimes the expression neutraceuticals or nutriceuticals are used as synonyms for nutraceuticals.

In one embodiment the nutraceuticals are selected from probiotics, prebiotics, synbiotics, amino acids, fatty acids, natural extracts, herbals, enzymes, lecithin, vitamins, minerals, butyric acid, omega 3, fish oil, algae oil, krill oil or mixtures thereof.

### Optional Top Coats and Sub Coats

Optionally the hard shell capsule can be additionally coated with a sub coat or a top coat or both.

A sub coat can be located between capsule and the coating layer, comprising the at least one polymer as disclosed. A sub coat has essentially no influence on the active ingredient release characteristics but may for instance improve the adhesion of the polymer coating layer. A sub coat is preferably essentially water-soluble, for instance it may consist of substances like HPMC as a film former. The average thickness of a sub coat layer is usually very thin, for example not more than 15 μm, preferably not more than 10 μm (0.1-1.0 mg/cm²). A sub coat or a top coat has not necessarily to be applied on the hard shell capsule in the pre-locked state.

A top coat can be located onto the coating layer, comprising the at least one polymer as disclosed. A top coat is also preferably water-soluble or essentially water-soluble. A top coat may have the function of colouring the pharmaceutical or nutraceutical form or protecting from environmental influences for instance from moisture during storage. The top coat can consist out of a binder, for instance a water-soluble polymer like a polysaccharide or HPMC, or a sugar compound like saccharose. The top coat can further contain pharmaceutically or nutraceutically acceptable excipients like pigments, plasticizers, emulsifiers or glidants in high amounts. The topcoat has essentially no influence on the release characteristics. A top coat can be applied on top of the pharmaceutical or nutraceutical dosage form comprising the polymer-coated hard shell capsule in the final-locked state as described herein. The average thickness of a top coat layer is usually very thin, for example not more than 15 μm, preferably not more than 10 μm (0.1-1.0 mg/cm²).

### Process for Preparing a Coated Hard Shell Capsule

Described is a process for preparing a polymer-coated hard shell capsule, suitable as container for biologically active ingredients, wherein the hard shell capsule comprises a body and a cap, wherein in the closed state the cap overlaps the body either in a pre-locked state or in a final-locked state, wherein the hard shell capsule is provided in the pre-locked state and coated, preferably spray-coated, with a coating solution, suspension or dispersion according to the present invention to create a coating layer which covers the outer surface of the hard shell capsule in the pre-locked state.

In a further process step the pre-locked hard shell capsule can be provided with a fill comprising at least one biologically active ingredient and is closed to the final-locked state.

In such a further process step the polymer-coated hard shell capsule in the pre-locked state can be opened, filled with a fill comprising a biologically active ingredient, and is closed in the final-locked state. This further process step is preferably performed in that the coated hard shell capsule in the pre-locked state is provided to a capsule-filling machine, which performs the opening, filling with a fill comprising at least one biologically active ingredient and closing of the polymer-coated hard shell capsule to the final-locked state.

This further process step results in a final-locked polymer-coated hard shell capsule, which is a container for at least one biologically active ingredient. The final-locked polymer-coated hard shell capsule, which as a container for at least one biologically active ingredient is a pharmaceutical or nutraceutical dosage form.

The dosage form preferably comprises a polymer-coated hard shell capsule in the final-locked state containing a fill comprising at least one biologically active ingredient, wherein the polymer-coated hard shell capsule comprises a coating layer according to the invention, where the coating layer covers the outer surface area of the capsule in the pre-locked state but not the overlapping area where the cap covers the body in the pre-locked state.

A coating suspension according to the present invention can contain an organic solvent, for instance acetone, iso-propanol or ethanol. The concentration of dry weight material in the organic solvent can be about from 5 to 50% by weight of polymer. A suitable spraying concentration can be about 5 to 25% by dry weight.

A coating suspension can be the dispersion according to the present invention in an aqueous medium, for instance water or a mixture of 80% by weight or more of water and 20% or less by weight of water-soluble solvents, such as acetone or isopropanol. A suitable concentration of dry weight material in the aqueous medium can be from about 5 to 50% by weigh. A suitable spraying concentration can be about 5 to 25% by dry weight.

The spray coating is preferably performed by spraying the coating solution or dispersion onto the pre-locked capsules in a drum coater or in a fluidized bed coating equipment.

### Process for Preparing a Fill for the Dosage Form

Suitable processes for preparing the fill for the pharmaceutical or nutraceutical dosage form are well known to a skilled person. A suitable pprocess for preparing the fill for the pharmaceutical or nutraceutical dosage form as disclosed herein can be by forming a core comprising the biologically active ingredient in the form of pellets by direct compression, compression of dry, wet or sintered granules, by extrusion and subsequent rounding off, by wet or dry granulation, by direct pelleting or by binding powders onto active ingredient-free beads or neutral cores or active ingredient-containing particles or pellets and optionally by applying coating layers in the form of aqueous dispersions or organic solutions in spray processes or by fluidized bed spray granulation.

### Capsule Filling Machine

The polymer-coated hard shell capsule is provided in the pre-locked state to a capsule-filling machine, which performs the steps of separating the body and the cap, filling the body with the fill and rejoining the body and the cap in the final-locked state.

The capsule filling machine used can be a capsule filling machine, preferably a fully automated capsule filling machine, that is capable to produce filled and closed capsules at a speed with an output of 1,000 or more filled and finally closed capsules per hour. Capsule filling machines, preferably fully automated capsule filling machines, are well known in the art and commercially available from several companies. A suitable capsule filling machine as used in the examples can be for instance ACG, model AFT Lab.

The capsule filling machine used can be preferably operated at a speed with an output of 1,000 or more, preferably 10,000 or more, 100,000 or more, 10,000 up to 500,000, filled and finally closed capsules per hour.

### Capsule Filling Machine General Operations

Before the capsule filling process, the capsule filling machine is provided with a sufficient number or amount of pre-coated hard shell capsules in the pre-locked state. The capsule filling machine is also provided with enough amounts of fill to be filled in during operation.

The hard shell capsules in the pre-locked state may fall by gravity into feeding tubes or chutes. The capsules can be uniformly aligned by mechanically gauging the diameter differences between the cap and the body. The hard shell capsules are then usually fed, in proper orientation, into a two-section housing or brushing.

The diameter of the upper bushing or housing is usually larger than the diameter of the capsule body bushing; thus, the capsule cap can be retained within an upper bushing while the body is pulled into a lower bushing by vacuum. Once the capsule is opened/the body and the cap are separated, the upper and lower housing or bushing are separated to position the capsule body for filling.

The open capsule body is then filled with the fill. Various types of filling mechanisms can be applied, with respect to the different fillings such as granules, powders, pellets or mini-tablets.

Capsule filling machines in general employ a variety of mechanisms to handle the various dosage ingredients as well as various numbers of filling stations. The dosing systems are usually based on volumetric or amounts of fills governed by the capsule size and capacity of the capsule body. The empty capsule manufacturers usually provide reference tables that indicate the volume capacity of their capsule body and the maximum fill weight for different capsule sizes based on the density of the fill material. After the filling, the body and the cap are rejoined by the machine in the final-locked state or position.

### Use/Method of Use/Method Steps

The process for preparing a polymer-coated hard shell capsule suitable as described herein can be understood as a method of use of a hard shell capsule comprising a body and a cap, wherein in the closed state the cap overlaps the body either in a pre-locked state or in a final-locked state, for preparing a polymer-coated hard shell capsule, suitable as container for pharmaceutical or nutraceutical biologically active ingredients, comprising the steps of
a) providing the hard shell capsule is provided in the pre-locked state and
b) spray-coating with a coating solution, suspension or dispersion comprising a polymer or a mixture of polymers to create a coating layer which covers the outer surface of the hard shell capsule in the pre-locked state.

The spray-coating can be preferably applied by using a drum coater equipment or a fluidized bed coating equipment. A suitable product temperature during the spray-coating process can be in the range from about 15 to 40, preferably from about 20 to 35° C. A suitable spray rate can be in the range from about 0.3 to 17.0, preferably 0.5 to 14 [g/min/kg]. After spray-coating a drying step is included.

The polymer-coated hard shell capsule in the pre-locked state can be opened in a step c), filled with a fill comprising a pharmaceutical or a nutraceutical biologically active ingredient in a step d), and is then closed in a step e) to the final-locked state.

Steps c) to e) can be performed manually or preferably supported by a suitable equipment, for instance a capsule-filling machine. Preferably, the coated hard shell capsule in the pre-locked state is provided to a capsule-filling machine, which performs the opening step c), the filling with a fill comprising a pharmaceutical or a nutraceutical biologically active ingredient in step d) and the closing of the capsule to the final-locked state in step e).

The selection of the processes in all their generic or specific features and embodiments as disclosed herein can be combined without restriction with any other generic or specific selections of materials or numerical features and embodiments as disclosed herein, such as polymers, capsule materials, capsule sizes, coating thicknesses, biologically active ingredients and any other embodiments as disclosed.

### Pharmaceutical or Nutraceutical Dosage Form

Disclosed is a pharmaceutical or nutraceutical dosage form comprising a polymer-coated hard shell capsule in the final-locked state containing a fill comprising a pharmaceutical or nutraceutical biologically active ingredient, wherein the polymer-coated hard shell capsule comprises a coating layer comprising at least one (meth)acrylate copolymer a), where the coating layer covers the outer surface area of the capsule in the pre-locked state. Since the outer surface area of the capsule in the pre-locked state is larger than outer surface area of the capsule in the final-locked state a part of the polymer coating layer is hidden or enclosed between the body and the cap of the hard shell capsule, which provides an efficient sealing.

### Items

In particular, the present invention refers to:
1. Process for preparing a polymer-coated hard shell capsule, suitable as container for pharmaceutical or nutraceutical biologically active ingredients, wherein the hard shell capsule comprises a body and a cap, wherein in the closed state the cap overlaps the body either in a pre-locked state or in a final-locked state, wherein the hard shell capsule is provided in the pre-locked state and coated, preferably spray-coated, with a coating solution, suspension or dispersion comprising or consisting of
   a) at least one (meth)acrylate copolymer a), comprising polymerized units of 5 to 25% by weight of methacrylic acid and 75 to 95% by weight of C1-to C4-alkylesters of methacrylic acid and/or C1-to C4-alkylesters of acrylic acid;
   b) 1 to 25, preferably 5 to 18, % by weight, based on the total weight of the at least one (meth)acrylate copolymer a), of at least one alkali or ammonium salt of a saturated aliphatic monocarboxylic acid having 10 to 30 carbon atoms;
   c) glycerol monostearate and/or glycerol distearate;
   d) at least one plasticizer selected from the group consisting of alkyl citrates, alkyl phthalates, alkyl sebacates, diethyl sebacate, dibutyl sebacate, glycerol, polyethylene glycols, and polypropylene glycols; and
   e) optionally at least one additive;
   to obtain a coated, preferably only at the outer surface, hard shell capsule in the pre-locked state.
2. Process according to item 1, wherein the base material of the body and the cap is selected from hydroxypropyl methyl cellulose, starch, gelatin, pullulan and a copolymer of a C1-to C4-alkylester of (meth)acrylic acid and (meth)acrylic acid, preferably is hydroxypropyl methyl cellulose.
3. Process according to any of the preceding items, wherein the at least one (meth)acrylate copolymer a) comprises an overall monomer composition by weight comprising polymerized units of 10 to 30% by weight methyl methacrylate, 50 to 70% by weight methyl acrylate and 5 to 15% by weight methacrylic acid.
4. Process according to any of the preceding items, wherein the at least one alkali or ammonium salt of a saturated aliphatic monocarboxylic acid having 10 to 30 carbon atoms is selected from alkali or ammonium salts of decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid and triacontanoic acid (melissic acid, C30) or mixtures thereof, wherein the alkali salt of the saturated aliphatic monocarboxylic acid is preferably at least one stearate, more preferably sodium stearate.
5. Process according to any of the preceding items, wherein glycerol monostearate and/or glycerol distearate are present in 1 to 10% by weight, preferably 2 to 7% by weight, based on the total weight of the at least one (meth)acrylate copolymer a).
6. Process according to any of the preceding items, wherein at least one further glidant, in addition to glycerol monostearate and/or glycerol distearate is present, preferably the at least one glidant
   i) is present in an amount of 3 to 75% by weight, based on the total weight of the at least one (meth)acrylate copolymer a) and/or
   ii) is selected from silica, ground silica, fumed silica, kaolin calcium silicate, magnesium silicate, colloidal silicone dioxide, talc, stearate salts selected from the group consisting of calcium stearate, magnesium stearate, and zinc stearate, sodium stearyl fumarate, starch, and stearic acid or mixtures thereof, preferably talc, magnesium stearate, and colloidal silicon dioxide or mixtures thereof.
7. Process according to any of the preceding items, wherein the at least one plasticizer
   i) is present in an amount of 0.5 to 40% by weight, based on the total weight of the at least one (meth)acrylate copolymer a) and/or
   ii) is selected from alkyl citrates, alkyl phthalates, and alkyl sebacates, glycerol, polyethylene glycols, propylene glycols or combinations thereof, preferably is selected from triethyl citrate (TEC), glycerol, polyethylene glycols, preferably having a number average molecular weight of 200 to 20,000 g/mol, or mixtures thereof.
8. Process according to any of the preceding items, wherein up to 400% by weight, preferably up to 200% by weight, more preferably up to 100% by weight or up to 50% by weight, or up to 30% by weight, or up to 15% by weight, or up to 5% by weight or up to 3% by weight, or up to 1% by weight, based on the total weight of the at least one (meth)acrylate copolymer a), of at least one additive are comprised; preferably selected from antioxidants, brighteners, emulsifiers, flavouring agents, flow aids, fragrances, penetration-promoting agents, pigments, polymers different from a), pore-forming agents or stabilizers, or combinations thereof.
9. Process according to item 8, wherein the at least one additive comprises at least one emulsifier, which is preferably
   i) present in an amount of 1.5 to 40% by weight, based on the total weight of the at least one (meth)acrylate copolymer a) and/or
   ii) a non-ionic emulsifier, preferably a non-ionic emulsifier having an HLB >10.
10. Process according to any of the preceding items, wherein the body and the cap are comprising encircling notches or dimples in the area where the cap overlaps the body, that allow the capsule to be closed by a snap-into-place mechanism either in the pre-locked state or in the final-locked state.
11. Process according to any of the preceding items, wherein the body comprises a tapered rim.
12. Process according to any of the preceding items, wherein the coating layer is applied in an amount of about 0.7 to 20 mg/cm², preferably 2 to 10, 4 to 8, 1.0 to 8, 1.5 to 5.5, or 1.5 to 4 mg/cm².
13. Process according to any of the preceding items, wherein the polymer-coated hard shell capsule in the pre-locked state is opened, filled with a fill comprising a pharmaceutical or a nutraceutical biologically active ingredient, and is closed to the final-locked state.
14. Process according to any of the preceding items, wherein the polymer-coated hard shell capsule in the pre-locked state is provided to a capsule-filling machine, which performs opening, filling with a fill comprising a pharmaceutical or a nutraceutical biologically active ingredient and closing to the final-locked state.
15. Polymer-coated hard shell capsule, obtained from a process according to any of items 1 to 14.
16. Use of the polymer-coated hard shell capsule according to item 15 for delayed or sustained release, preferably delayed release, more preferably for delayed or sustained release for colon delivery.

### EXAMPLES

### Preparation of compositions

Used Materials:

EUDRAGUARD® biotic dispersion is a 30 wt. % aqueous dispersion of (meth)acrylic copolymer containing 25 wt. % methyl methacrylate units, 65 wt. % methyl acrylate units, 10 wt. % methacrylic acid units (manufacturer: Evonik Nutrition & Care GmbH).

Sodium stearate was purchased from Sigma-Aldrich.

Glycerol monostearate (GMS Imwitor 900 K) was purchased from Cremer.

Polysorbate 80 (Tween 80) was purchased from Sigma Aldrich.

Polyethylene glycol 6000 (PEG 6000) was purchased from Merck.

Triethyl citrate (TEC) was purchased from Merck.

Glycerol was purchased from Merck.

### Formulations

In tables 1 and 2 the components are indicated in weight %. All components, except EUDRAGUARD® biotic (30 wt.-% polymer content), are indicated as pure substance.

TABLE 1

| | Formulation | Formulation | Formulation | Formulation |
|---|---|---|---|---|
| | 1 | 2 | 3 | 4 |
| Components | (comparative) | (inventive) | (inventive) | (inventive) |
| EUDRA - | 100 | 100 | 100 | 100 |
| GUARD ® | | | | |
| biotic | | | | |
| Sodium Stearate | | 1.2 | 1.2 | 1.2 |
| Triethyl citrate | 1.5 | 1.5 | | |
| PEG 6000 | | | 1.5 | |
| Glycerol | | | | 1.5 |
| Glycerol | 1.5 | 1.5 | 1.5 | 1.5 |
| monostearate | | | | |
| Polysorbate 80 | 0.6 | 0.6 | 0.6 | 0.6 |
| Water | 232 | 232 | 232 | 232 |

TABLE 2

| | Formulation 5 | Formulation 6 |
|---|---|---|
| Components | (inventive) | (comparative) |
| EUDRAGUARD ® biotic | 100 | 100 |
| Sodium Stearate | 1.2 | 1.2 |
| Triethyl citrate | 1.05 | 1.05 |
| Glycerol | 0.45 | 0.45 |
| Glycerol monostearate | 1.5 | 3.0 |
| Polysorbate 80 | 0.6 | 1.2 |
| Water | 232 | 230 |

Water, GMS, polysorbate 80, sodium stearate and the respective plasticizer were mixed, heated up to 60° C. and homogenized with a high shear mixer e.g. Ultra Turrax. After cooling down to 25° C. while stirring the mixture was added to EUDRAGUARD® biotic.

### Example 1 (Not According to the Invention)

In a Hüttlin Fluidized bed coater formulation 2 was applied on diprophyline pellets. Coating temperature was 30-35° C. Coating amount was 14% polymer.

The drug release was determined according to USP (Paddle) at increasing pH values of the media.

### Example 2

In an O'Hara Drumcoater formulation 2 was applied on preclosed HPMC capsules at 35-40° C.

Applied polymer amount was 3 mg/cm².

The capsules were filled with diprophyline pellets and closed.

The drug release was determined according to USP (Paddle) at increasing pH values of the media.

### Example 3 (Not According to the Invention)

In an O'Hara Drumcoater formulation 1 was applied on preclosed HPMC capsules at 35-40° C.

Applied polymer amount was 3 mg/cm².

The capsules were filled with diprophyline pellets and closed.

The drug release was determined according to USP (Paddle) at increasing pH values of the media.

### Example 4

In an O'Hara Drumcoater formulation 3 was applied on preclosed HPMC capsules at 35-40° C.

Applied polymer amount was 3 mg/cm².

The capsules were filled with diprophyline pellets and closed.

The drug release was determined according to USP (Paddle) at increasing pH values of the media.

### Example 5

In an O'Hara Drumcoater formulation 4 was applied on preclosed HPMC capsules at 35-40° C.

Applied polymer amount was 3 mg/cm².

The capsules were filled with diprophyline Pellets and closed.

The drug release was determined according to USP (Paddle) at increasing pH values of the media.

### Example 6

In an O'Hara Drumcoater formulation 5 was applied on preclosed HPMC capsules at 35-40° C.

Applied polymer amount was 3 mg/cm².

The capsules were filled with diprophyline Pellets and closed.

The drug release was determined according to USP (Paddle) at increasing pH values of the media.

### Example 7 Comparative

In an O'Hara Drumcoater formulation 6 was applied on preclosed HPMC capsules at 35-40° C.

Applied polymer amount was 3 mg/cm².

The capsules were filled with diprophyline Pellets and closed.

The drug release was determined according to USP (Paddle) at increasing pH values of the media.

Drug release comparison capsules/pellets

| | | Pellets Example 1 | Capsules Example 2 |
|---|---|---|---|
| Time | pH- | (comparative) | (inventive) |
| [Min] | value | Drug release [%] | Drug release [%] |
| 0 | 1 | 0.00 | 0 |
| 60 | 1 | 1 | 0 |
| 120 | 1 | 2 | 0 |
| 150 | 6.8 | 71 | 0 |
| 165 | 6.8 | 100 | 0 |
| 180 | 6.8 | 100 | 0 |
| 195 | 7.2 | 100 | 0 |
| 210 | 7.2 | 100 | 13 |
| 225 | 7.2 | 100 | 57 |
| 240 | 7.2 | 100 | 88 |
| 300 | 7.2 | 100 | 100 |

Drug release Capsules

| | pH- | Example 3 | Example 2 | Example 4 | Example 5 |
|---|---|---|---|---|---|
| min | value | (comparative) | (inventive) | (inventive) | (inventive) |
| 0 | 1 | 0 | 0 | 0 | 0 |
| 60 | 1 | 0 | 0 | 0 | 0 |
| 120 | 1 | 0 | 0 | 0 | 0 |
| 150 | 6.8 | 0 | 0 | 0 | 0 |
| 180 | 6.8 | 0 | 0 | 0 | 1 |
| 185 | 7.2 | 0 | 0 | 0 | 3 |
| 195 | 7.2 | 0 | 0 | 8 | 16 |
| 200 | 7.2 | 0 | 2 | 23 | 25 |
| 210 | 7.2 | 2 | 13 | 45 | 47 |
| 220 | 7.2 | 8 | 43 | 60 | 75 |
| 225 | 7.2 | 14 | 57 | 67 | 84 |
| 240 | 7.2 | 37 | 88 | 87 | 100 |
| 300 | 7.2 | 98 | 100 | 100 | 100 |
| 303 | 7.2 | 100 | 100 | 100 | 100 |
| | pH- | Example 6 | Example 7 | | |
| min | value | (inventive) | (comparative) | | |
| 0 | 1 | 0 | 0 | | |
| 60 | 1 | 0 | 0 | | |
| 120 | 1 | 0 | 0 | | |
| 150 | 6.8 | 0 | 0 | | |
| 180 | 6.8 | 0 | 0 | | |
| 185 | 7.2 | 0 | 0 | | |
| 195 | 7.2 | 5 | 1 | | |
| 200 | 7.2 | 10 | 4 | | |
| 210 | 7.2 | 19 | 7 | | |
| 220 | 7.2 | 34 | 18 | | |
| 225 | 7.2 | 44 | 22 | | |
| 240 | 7.2 | 81 | 48 | | |
| 300 | 7.2 | 100 | 100 | | |
| 303 | 7.2 | 100 | 100 | | |

1. Process for preparing a polymer-coated hard shell capsule, suitable as container for pharmaceutical or nutraceutical biologically active ingredients, wherein the hard shell capsule comprises a body and a cap, wherein in the closed state the cap overlaps the body either in a pre-locked state or in a final-locked state, wherein the hard shell capsule is provided in the pre-locked state and coated with a coating solution, suspension or dispersion comprising or consisting of
a) at least one (meth)acrylate copolymer a), comprising polymerized units of 5 to 25% by weight of methacrylic acid and 75 to 95% by weight of C1-to C4-alkylesters of methacrylic acid and/or C1-to C4-alkylesters of acrylic acid;
b) 1 to 25, preferably 5 to 18, % by weight, based on the total weight of the at least one (meth)acrylate copolymer a), of at least one alkali or ammonium salt of a saturated aliphatic monocarboxylic acid having 10 to 30 carbon atoms;
c) glycerol monostearate and/or glycerol distearate;
d) at least one plasticizer selected from the group consisting of alkyl citrates, alkyl phthalates, alkyl sebacates, diethyl sebacate, dibutyl sebacate, glycerol, polyethylene glycols, trialkyl citrate and polypropylene glycols; and
e) optionally at least one additive;
to obtain a coated, preferably only at the outer surface, hard shell capsule in the pre-locked state.

2. The process according to claim 1, wherein the base material of the body and the cap is selected from hydroxypropyl methyl cellulose, starch, gelatin, pullulan and a copolymer of a C1-to C4-alkylester of (meth)acrylic acid and (meth)acrylic acid.

3. The process according to claim 1, wherein the at least one (meth)acrylate copolymer a) comprises an overall monomer composition by weight comprising polymerized units of 10 to 30% by weight methyl methacrylate, 50 to 70% by weight methyl acrylate and 5 to 15% by weight methacrylic acid.

4. The process according to claim 1 wherein the at least one alkali or ammonium salt of a saturated aliphatic monocarboxylic acid having 10 to 30 carbon atoms is selected from alkali or ammonium salts of decanoic acid (capric acid, C10), undecanoic acid, dodecanoic acid (lauric acid, C12), tridecanoic acid, tetradecanoic acid, pentadecanoic acid, hexadecenoic acid (palmitic acid, C16), heptadecanoic acid, octadecanoic acid (stearic acid, C18), nonadecanoic acid, eicosanoic acid (arachidic acid, C20), heneicosanoic acid (behenic acid, C22), docosanoic acid, tricosanoic acid, pentacosanoic acid, hexacosanoic acid (ceratic acid), heptacosanoic acid, octacosanoic acid, nonacosanoic acid and triacontanoic acid (melissic acid, C30) or mixtures thereof, wherein the alkali salt of the saturated aliphatic monocarboxylic acid is preferably at least one stearate, more preferably sodium stearate.

5. The process according to claim 1, wherein glycerol monostearate and/or glycerol distearate are present in 1 to 10% by weight, preferably 2 to 7% by weight, based on the total weight of the at least one (meth)acrylate copolymer a).

6. The process according to claim any of the preceding claims, wherein at least one further glidant, in addition to glycerol monostearate and/or glycerol distearate is present, preferably the at least one glidant
i) is present in an amount of 3 to 75% by weight, based on the total weight of the at least one (meth)acrylate copolymer a) and/or
ii) is selected from silica, ground silica, fumed silica, kaolin calcium silicate, magnesium silicate, colloidal silicone dioxide, talc, stearate salts selected from the group consisting of calcium stearate, magnesium stearate, and zinc stearate, sodium stearyl fumarate, starch, and stearic acid or mixtures thereof.

7. The process according to claim 1, wherein the at least one plasticizer
i) is present in an amount of 0.5 to 40% by weight, based on the total weight of the at least one (meth)acrylate copolymer a) and/or
ii) is selected from alkyl citrates, alkyl phthalates, and alkyl sebacates, glycerol, polyethylene glycols, propylene glycols or combinations thereof, preferably is selected from triethyl citrate (TEC), glycerol, polyethylene glycols, preferably having a number average molecular weight of 200 to 20,000 g/mol, or mixtures thereof.

8. The process according to claim lany of the preceding claims, wherein up to 400% by weight, based on the total weight of the at least one (meth)acrylate copolymer a) of at least one additive are comprised.

9. The process according to claim 8, wherein the at least one additive comprises at least one emulsifier, which is preferably
i) present in an amount of 1.5 to 40% by weight, based on the total weight of the at least one (meth)acrylate copolymer a) and/or
ii) a non-ionic emulsifier.

10. The process according to claim 1, wherein the body and the cap are comprising encircling notches or dimples in the area where the cap overlaps the body, that allow the capsule to be closed by a snap-into-place mechanism either in the pre-locked state or in the final-locked state.

11. The process according to claim 1, wherein the body comprises a tapered rim.

12. The process according to claim 1, wherein the coating layer is applied in an amount of about 0.7 to 20 mg/cm².

13. The process according to claim 1, wherein the trialkyl citrate is triethyl citrate.

14. A polymer-coated hard shell capsule, obtained from a process according to claim 1.

15. Use of the polymer-coated hard shell capsule according to claim 14 for delayed or sustained release.
